(19) United States
(12) Patent Application Publication
(10) Pub. No.: US 2005/0013778 A1
(43) Pub. Date: Jan. 20, 2005

(54) Methods and compositions for predicting the response to a therapeutic regimen in a subject having a disease associated with cell death

(75) Inventors: Allan M. Green, Cambridge, MA (US); Neil Steinmetz, Atlantis, FL (US)

Correspondence Address: LAHIVE & COCKFIELD, LLP., 28 STATE STREET, BOSTON, MA, 02109, US

(73) Assignee: Theseus Imaging Corporation, Boston, MA (US)

(21) Appl. No.: 10/819,890

(22) Filed: Apr. 6, 2004

Related U.S. Application Data
(63) Continuation-in-part of application No. 10/114,927, filed on Apr. 3, 2002.
(60) Provisional application No. 60/281,277, filed on Apr. 3, 2001.

(51) Int. Cl. A61K 49/00; A61K 39/395; A61K 33/24

(52) U.S. Cl. 424/9.6; 424/144.1; 424/145.1; 424/649

(57) ABSTRACT

The present invention provides methods and compositions for predicting the response to a therapeutic regimen in a subject having a disease associated with cell death.

### RELATED APPLICATIONS

This application is a continuation-in-part and claims priority to U.S. patent application Ser. No. 10/114,927 filed Apr. 3, 2002, the contents of which are incorporated herein by reference. This application also claims priority to U.S. Provisional Patent Application No. 60/281,277 filed Apr. 3, 2001.

### BACKGROUND OF THE INVENTION

Apoptosis and Necrosis

Apoptosis refers to “programmed cell death” whereby the cell executes a “cell suicide” program. It is now thought that the apoptosis program is evolutionarily conserved among virtually all multicellular organisms, as well as among all the cells in a particular organism. Further, it is believed that in many cases, apoptosis may be a “default” program that must be actively inhibited in healthy surviving cells.

The decision by a cell to submit to apoptosis may be influenced by a variety of regulatory stimuli and environmental factors (Thompson, 1995). Physiological activators of apoptosis include tumor necrosis factor (TNF), Fas ligand, transforming growth factor β, the neurotransmitters glutamate, dopamine, N-methyl-D-asparate, withdrawal of growth factors, loss of matrix attachment, calcium and glucocorticoids. Damage-related inducers of apoptosis include heat shock, viral infection, bacterial toxins, the oncogenes myc, rel and E1A, tumor suppressor p53, cytolytic T-cells, oxidants, free radicals and nutrient deprivation (antimetabolites). Therapy-associated apoptosis inducers include gamma radiation, UV radiation and a variety of chemotherapeutic drugs, including cisplatin, doxorubicin, bleomycin, cytosine arabinoside, nitrogen mustard, methotrexate and vincristine. Toxin-related inducers or apoptosis include ethanol and d-amyloid peptide.

Apoptosis can have particularly devastating consequences when it occurs pathologically in cells that do not normally regenerate, such as neurons. Because such cells are not replaced when they die, their loss can lead to debilitating and sometimes fatal dysfunction of the affected organ. Such dysfunction is evidenced in a number of neurodegenerative disorders that have been associated with increased apoptosis, including Alzheimer's disease, Parkinson's disease, amyotrophic lateral sclerosis, retinitis pigmentosa and cerebellar degeneration.

The consequences of undesired apoptosis can be similarly devastating in other pathologies as well, including ischemic injury, such as typically occurs in cases of myocardial infarction, reperfusion injury and stroke. In particular, apoptosis is believed to play a central role in very delayed infarction after mild focal ischemia (Du, et al., 1996). Additional diseases associated with increased apoptosis include, but are not limited to, the following: AIDS; myelodysplatic syndromes, such as aplastic anemia; and toxin induced liver disease, including damage due to excessive alcohol consumption.

Necrosis is the localized death of cells or tissue due to causes other than apoptosis (i.e., other than the execution of the cell's intrinsic suicide program). Necrosis can be caused by traumatic injury, bacterial infection, acute hypoxia and the like. There is some overlap between the two types of cell death, in that some stimuli can cause either necrosis or apoptosis or some of both, depending on the severity of the injury.

Asymmetry of Biological Membranes

It is generally believed that biological membranes are asymmetric with respect to specific membrane phospholipids. In particular, the outer leaflet of eukaryotic plasma membranes is formed predominantly with the cholinephospholipids, such as sphingomyelin and phosphatidylcholine (PC), whereas the inner leaflet contains predominantly aminophospholipids, such as phosphatidylserine (PS) and phosphatidylethanolamine (PE). This asymmetry is thought to be maintained by the activity of an adenosine triphosphate (ATP)-dependent aminophospholipid translocase, which selectively transports PS and PE between bilayer leaflets (Seigneuret and Devaux, 1984). Other enzymes thought to be involved in the transport of phospholipids between leaflets include ATP-dependent floppase (Connor, et al., 1992) and lipid scramblase (Zwaal, et al., 1993).

Although asymmetry appears to be the rule for normal cells, the loss of such asymmetry is associated with certain physiological, as well as pathogenic, processes. For example, it has been recognized that membrane asymmetry, detected as appearance of PS on the outer leaflet of the plasma membrane (“PS exposure”), is one of the earliest manifestations of apoptosis, preceding DNA fragmentation, plasma membrane blebbing, and loss of membrane integrity (Martin, et al., 1995; Fadok, e! al., 1992).

Similar re-orientation has been observed in sickle cell disease (Lane, et al., 1994)″B-thalassemia (Borenstain-Ben Yashar, et al., 1993), platelet activation, and in some mutant tumor cell lines with defective PS transport. A gradual appearance of PS on the outer leaflet has also been observed to occur in aging red blood cells (Tait and Gibson, 1994). When the PS exposure on such cells reaches a threshold level, the cells are removed from circulation by macrophages (Pak and Fidler, 1991). All of the above conditions proximately culminate in the death of the affected cells (i.e., cells with significant PS exposure).

It will be appreciated that PS exposure is a component in both apoptosis and necrosis. Its role in the initial stages of apoptosis is summarized above. Once the apoptotic cell has reached the terminal stages of apoptosis (i.e., loss of membrane integrity), it will be appreciated that the PS in both plasma membrane leaflets will be “exposed” to the extracellular milieu. A similar situation exists in cell death by necrosis, where the loss of membrane integrity is either the initiating factor or occurs early in the necrotic cell death process; accordingly, such necrotic cells also have “exposed” PS, since both plasma membrane leaflets are “exposed”.

Annexin

Annexin V is normally found in high levels in the cytoplasm of a number of cells including placenta, Lymphocytes, monocytes, biliary and renal (cortical) tubular epithelium. Although the physiological function of annexins has not been fully elucidated, several properties of annexins make them useful as diagnostic and/or therapeutic agents. In particular, it has been discovered that annexins possess a very high affinity for anionic phospholipid surfaces, such as a membrane leaflet having an exposed surface of phosphatidylserine (PS).

### SUMMARY OF THE INVENTION

The present invention provides methods and compositions for imaging cell death in vivo, as well as methods and compositions for tumor radiotherapy and phototherapy. The present invention is based, at least in part, on the discovery that the combination of an annexin with a contrast agent allows for the efficient and effective detection of cells undergoing cell death using magnetic reasonance imaging. The present invention is also based, at least in part, on the discovery that the combination of an annexin with an optically active molecule, such as a fluorescent dye, allows for the efficient and effective detection of cells undergoing cell death by optical imaging. Finally, the present invention is based, at least in part, on the discovery that administering a composition comprising an annexin coupled with a therapeutic radioisotope to a tumor bearing subject that has been treated with chemotherapeutic agent, allows for the specific and enhanced delivery of the radiation carried by the annexin-therapeutic radioisotope composition to the tumor site.

Accordingly, the present invention provides a magnetic reasonance imaging composition which includes an annexin, e.g., annexin V, coupled to a contrast agent, such as a paramagnetic agent (e.g., a gadolinium-chelating group complex, such as gadolinium-diethylenetriamine penta-acetic acid, or a lanthanum chelating group complex) or a superparamagnetic agent (e.g., a metal oxide, such as Fe, Co, Ni, Cu, Zn, As, Se, Mo, Tc, Ru, Rh, Pd, Ag, Cd, In, Sn, Re, Os, Ir, Pt, Au, Hg, Tl, Pb, Bi, Po, or At oxide). The metal oxide is preferably coated with a polymer, e.g., dextran or variants thereof. The annexin may be coupled to the contrast agent directly or indirectly.

In another aspect, the present invention provides compositions comprising an annexin, e.g., annexin V, coupled to a contrast agent, such as a polymer coated metal oxide, and a radioisotope, e.g., a diagnostic or therapeutic radioisotope. Such compositions are suitable for both MRI and nuclear medicine imaging. For example, the composition may include annexin V coupled to a contrast agent and a radioisotope (linked to the annexin via hydrazino nicotinamide (HYNIC)). In one embodiment, the annexin may be coupled to a carrier that is cleared or metabolized by a desirable route. Examples of such carriers include, but are not limited to, dextran particles or colloidal particles or metal oxide particles, such as superparamagnetic iron oxide particles (which are typically phagocytosed in the liver).

In another aspect, the present invention provides a method for the in vivo imaging of cell death, e.g., cell death caused by apoptosis, in a mammalian subject, for example, in an organ of a mammalian subject or a portion thereof (e.g., brain, heart, liver, lung, pancreas, colon) or a gland of a mammalian subject or a portion thereof (e.g., prostate or mammary gland). The method includes administering to the subject a magnetic reasonance imaging composition comprising annexin coupled to a contrast agent; and obtaining a magnetic reasonance image, wherein said image is a representation of cell death in the mammalian subject. In one embodiment, the magnetic reasonance image is obtained 5, 10, 15, 20, 25, 30, 35, 40, 45, 50, 55, 60, 65, 70, 75, 80, 85, 90, 95, 100, 105, 110, 115, or 120 minutes after the administration of the magnetic reasonance imaging composition to the subject. In another embodiment, the magnetic reasonance image is obtained about 12-30, 15-25, 20-25, or 20-30 hours after the administration of the magnetic reasonance imaging composition to the subject. Ranges intermediate to the above recited values are also intended to be part of this invention. For example, ranges using a combination of any of the above recited values as upper and/or lower limits are intended to be included. In a preferred embodiment, the magnetic reasonance image is obtained at a plurality of time points, thereby monitoring changes in the number of cells undergoing cell death or monitoring changes in the location of cells undergoing cell death.

The magnetic resonance imaging composition may be administered at a concentration of 1-1000 μg protein/kg, 1-900 μg protein/kg, 1-800 μg protein/kg, 1-700 μg protein/kg, 1-600 μg protein/kg, 1-500 μg protein/kg, 1-400 μg protein/kg, 1-300 μg protein/kg, 1-200 μg protein/kg, 1′-1100 μg protein/kg, 1-50 μg protein/kg, or 1-20 μg protein/kg. In another embodiment, the magnetic reasonance imaging composition is administered intravenously, intraperitoneally, intrathecally, intrapleurally, intralymphatically, or intramuscularly.

In a further aspect, the present invention provides an optical imaging composition which includes an annexin, e.g., annexin V, coupled to a biologically compatible and optically active molecule, such as a fluorescent dye like fluorescein, which can be visualized during optical evaluations such as endoscopy, brochoscopy, peritonoscopy, direct visualization, surgical microscopy and retinoscopy. Moreover, by the appropriate choice of optically active molecule, an annexin-optically active molecule combination may be useful in photodynamic therapy (PDT), a novel approach for the treatment of cancer and other diseases, such as macular degeneration, which may be used as a primary or adjunctive therapeutic modality. In the present invention, PDT works by exposing an annexin molecule linked to a photosensitizing drug to specific wavelengths of light in the presence of oxygen. When this reaction occurs, the normally innocuous photosensitizing molecule becomes cytotoxic via an activated species of oxygen, known as “singlet oxygen.” The ability of annexin to localize at sites of tumor cell apoptosis makes this an ideal drug to use in combination with anti-cancer treatment which leads to apoptosis or necrosis of tumor cells. The temporal introduction of the annexin-photosensitizing drug after induction of tumor cell apoptosis or necrosis creates a circumstance for differential localization of the annexin-photosensitizing molecule combination at the tumor site, providing the opportunity for additional tumor cell killing using appropriate light exposure. Typically, laser energy, delivered to the diseased tissue, e.g., cancer site, directly or through a fiberoptic device, chemically activates the drug and creates a toxic form of oxygen which destroys the cancerous cells with minimal damage to healthy cells. Examples of optically active agents which could be used in PDT when linked to annexin include PHOTOFRIN®, Lutrin, ANTRIN®, FOSCAN®, aminolevulinic acid, aluminum (III) phthalocyanine tetrasulfonate, Hypericin, verteporfin, and methylene blue dye. Among the possible targets for PDT are tumors of the brain, head and neck, breast, esophagus, lung, pleural cavity, ovary, abdominal cavity, bladder, prostate, cervix, skin, peritoneal cavity, eye and aerodigestive system.

In yet another aspect, the present invention provides a method for imaging cell death in a mammalian subject in vivo by administering to the subject an optical imaging composition comprising annexin coupled to an optically active molecule; illuminating the subject with a light source; and visually monitoring the presence of the optical imaging composition in the subject, thereby obtaining an image, wherein the image is a representation of cell death in the mammalian subject.

In another aspect, the present invention provides a composition comprising an annexin, e.g., annexin V, coupled with a therapeutic radioisotope, e.g., ¹⁰³Pd, ¹⁸⁶Re, ¹⁸⁸Re, ⁹⁰Y, ¹⁵³Sm, ¹⁵⁹Gd, ¹⁶⁶Ho or ¹⁷⁷Lu. The therapeutic radioisotope and the annexin may be coupled at a ratio of 1:1, 1.1:1, 1.2:1, 1.3:1, 1.4:1, 1.5:1, 1.6:1, 1.7:1, 1.8:1, 1.9:1, or 2:1 (therapeutic radioisotope:annexin). Ranges of values using a combination of any of the above recited values as upper and/or lower limits are intended to be included in the present invention.

In a further aspect, the present invention provides a method of tumor radiotherapy by administering to a mammalian subject having a tumor an effective tumor reducing amount of a composition comprising an annexin coupled with a therapeutic radioisotope. The foregoing method may be used in conjunction with total body irradiation or targeted external irradiation and/or a treatment employing at least one chemotherapeutic agent (e.g., dimethyl busulfan, cyclophosphamide, bischloroethyl nitrosourea, cytosine arabinoside, or 6-thioguanine). In addition, the method may be used in conjunction with biologically active anti-cancer agents and apoptosis inducing agents such as TNF, TRAIL or Fas or with antibodies, small molecules or pharmacophores which bind these receptors and also induce apoptosis.

In another aspect, the present invention features a method of tumor radiotherapy, which includes treating a subject having a tumor with a chemotherapeutic agent and subsequently administering to the subject an effective tumor reducing amount of a composition comprising an annexin coupled with a therapeutic radioisotope.

The timing of the administration of the annexin coupled with a therapeutic agent is critical to the effectiveness of the therapeutic intervention. The modified annexin should be administered at a time which assures its bioavailability at times of apoptosis or necrosis of the target tissue. Diagnostic imaging studies using radiolabeled annexin V indcate that the administration of a therapeutically modified annexin preferably should be within 24 hours of the completion of a course of chemotherapy of lymphoma with multiple antimetabolite drugs (so-called, CHOP or MOPP therapy) to optimize the availability of annexin localization in the damaged tumor. Optimal time of administration may be within 72 hours of chemotherapeutic treatment of solid tumors such as breast cancer, lung cancer or sarcoma as shown by imaging studies in patients. Use of a diagnostic imaging agent, such as radiolabeled annexin, to determine the extent of apoptosis may be used to qualify patients for administration of therapeutically modified annexin and to determine the optimal dose of therapeutically modified annexin.

In another aspect, the present invention features a method for predicting the response to a therapeutic regimen in a subject having a disease associated with cell death, e.g., a subject having a tumor. The method includes administering to the subject an annexin or fragment thereof which is detectably labeled, and detecting the localization of the annexin or fragment thereof within the subject, wherein the presence of the annexin or fragment thereof in the region of cell death is indicative of a positive response by the subject to a therapeutic regimen.

In yet another aspect, the present invention features a method for predicting the response to a therapeutic regimen in a subject having a disease associated with cell death by first administering to the subject the therapeutic regimen, followed by administering to the subject an annexin or fragment thereof which is detectably labeled, and then detecting a change in the uptake of the annexin or fragment thereof by the area of cell death over time, wherein a change in the uptake of the annexin or fragment thereof by the area of cell death over time is indicative of a positive response by the subject to the therapeutic regimen. The change may be an increase or a decrease in the uptake of the annexin or fragment thereof by the area of cell death over time. In one embodiment, the change may be detected by comparing the uptake of the annexin or fragment thereof by the area of cell death before and after the administration of the therapeutic regimen. In another embodiment, the change may be detected by comparing the uptake of the annexin or fragment thereof by the area of cell death at different time points after the administration of the therapeutic regimen.

Other features and advantages of the invention will be apparent from the following detailed description, and from the claims.

### BRIEF DESCRIPTION OF THE DRAWINGS

FIG. 1 depicts the attachment of Annexin V to an iron oxide coated with the non-polymer DMSA.

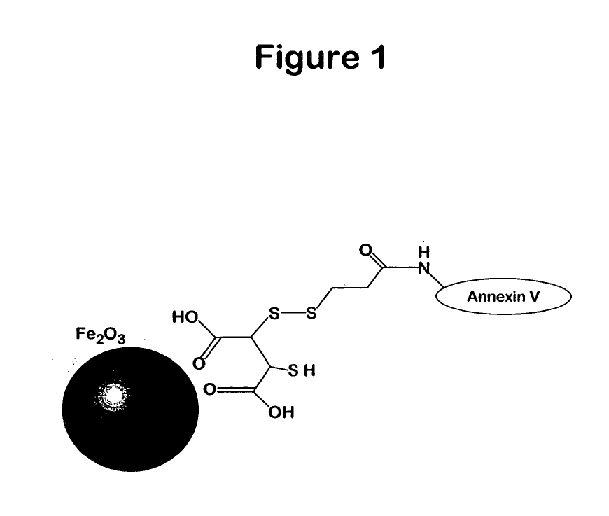

FIG. 2 depicts the attachment of Annexin V to a polymer coated magnetic iron oxide.

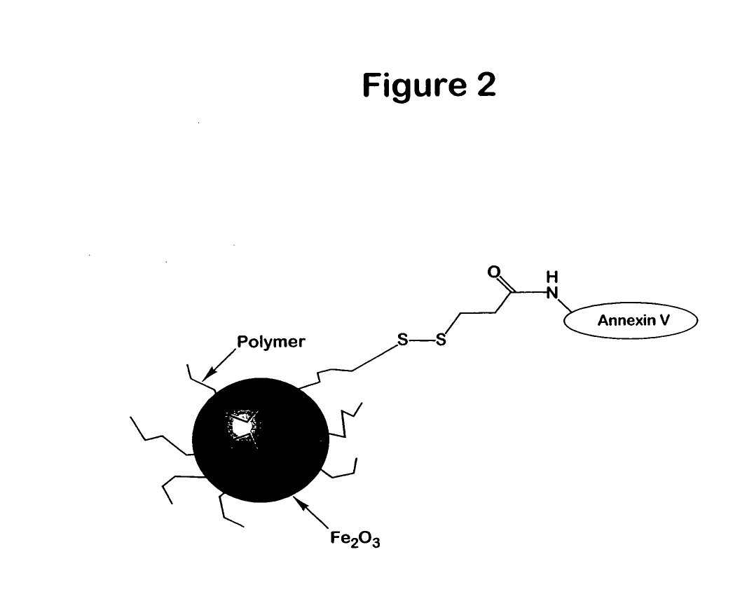

### DETAILED DESCRIPTION OF THE INVENTION

The present invention provides methods and compositions for imaging cell death in vivo, as well as methods and compositions for tumor radiotherapy. The present invention is based, at least in part, on the discovery that the combination of an annexin with a contrast agent allows for the efficient and effective detection of cells undergoing cell death using magnetic reasonance imaging. The present invention is also based, at least in part, on the discovery that the combination of an annexin with an optically active molecule, such as a fluorescent dye, allows for the efficient and effective detection of cells undergoing cell death by optical imaging.

Finally, the present invention is based, at least in part, on the discovery that administering a composition comprising an annexin coupled with a therapeutic radioisotope to a tumor bearing subject that has been treated with chemotherapeutic agent, allows for the specific and enhanced delivery of the radiation carried by the annexin-therapeutic radioisotope composition to the tumor site. Without intending to be limited by mechanism, it is believed that the administration of the chemotherapeutic agent will cause apoptosis or necrosis at the site of the tumor, thereby allowing the annexin-therapeutic radioisotope complex to be specifically targeted to the site of the tumor and delivering the radiation, which will kill the cell to which the annexin binds, as well as neighboring cells.

Accordingly, the present invention provides a magnetic reasonance imaging composition which includes an annexin or a fragment thereof, e.g., annexin V or a fragment thereof, coupled (either directly or indirectly) to a contrast agent.

As used herein, the term “annexin” is intended to include, but not limited to, each member of the annexin family of proteins (e.g., annexin V) annexin fragments; annexin derivatives; or peptides, peptidomimetics or small molecules that mimic the phospholipid binding domain of annexin. In a preferred embodiment, according to the invention, annexin V is utilized. Annexin V is one of the most abundant annexins. Furthermore, annexin V is conveniently produced from natural or recombinant sources. Lastly, annexin V has a high affinity for phospholipid membranes. Human annexin V has a molecular weight of 36 kd and a high affinity (kd=7 mmol/L) for phosphatidylserine. In particular, annexin V domain 1 has a high affinity for phosphatidylserine. The sequence of human annexin V can be obtained from GenBank under accession numbers U05760-U05770. In an alternative embodiment, annexin fragments (e.g., annexin V fragments) may be used to practice the invention. Preferably, the annexin fragment includes the conserved domain 1 of annexin V as is well known in the art (Montaville et al., 2002). In yet another embodiment, annexin derivatives may be used in practice of the invention. Examples of annexin derivatives (e.g., annexin V derivatives) for use in the methods described herein are disclosed in PCT Publication No. WO 00/20453, incorporated herein by reference. In an alternative embodiment, small molecules, peptides or peptidomimetics that mimic the phospholipid binding domain of annexin may be utilized. For example, small molecules, peptides or peptidomimetics that mimic domain 1 of annexin V may be used.

As used herein, a “contrast agent” is intended to include any agent that is physiologically tolerable and capable of providing enhanced contrast for magnetic reasonance imaging. Contrast agents typically have the capability of altering the response of a tissue to magnetic fields. Contrast agents include paramagnetic agents, e.g., a gadolinium-chelating group complex, such as gadolinium-diethylenetriamine penta-acetic acid, or a manganese chelating group complex; or biologically compatible superparamagnetic agents such as iron oxide. Contrast agents, such as those described in U.S. Pat. No. 4,687,658; U.S. Pat. No. 5,314,680; and U.S. Pat. No. 4,976,950 are intended to be used in preparing the compositions of the present invention. Contrast agents are commercially available (e.g., the gadolinium chelate Prohance™ is available from Squibb and the gadolinium chelate Dotarem™ is available from Guerbet).

A suitable contrast agent must preferably be biocompatible, e.g., non-toxic, chemically stable, not absorbed by the body or reactive with a tissue, and eliminated from the body within a short time. In one embodiment, the contrast agent may be coupled to a carrier that is cleared or metabolized by a desirable route. Examples of such carriers include, but are not limited to, dextran particles or colloidal particles (which are typically phagocytosed in the liver).

In another aspect, the present invention provides a method for the in vivo imaging of cell death, e.g., cell death caused by apoptosis, in a mammalian subject. The method includes administering to the subject a magnetic reasonance imaging composition comprising annexin coupled to a contrast agent; and obtaining a magnetic reasonance image, wherein said image is a representation of cell death in the mammalian subject.

As used herein, the term “cell death” includes the processes by which mammalian cells die. Such processes include apoptosis (both reversible and irreversible) and processes thought to involve apoptosis (e.g., cell senescence), as well as necrosis. “Cell death” is used herein to refer to the death or imminent death of nucleated cells (e.g., neurons, myocytes, hepatocytes and the like) as well as to the death or imminent death of anucleate cells (e.g., red blood cells, platelets, and the like). Cell death is typically manifested by the exposure of PS on the outer leaflet of the plasma membrane.

As used herein, the term “subject” includes warm-blooded animals, preferably mammals, including humans. In a preferred embodiment, the subject is a primate. In an even more preferred embodiment, the subject is a human. Cell death may be imaged or detected in, for example, an organ of a subject or a portion or specimen thereof (e.g., brain, heart, liver lung, pancreas, colon) or a gland of a subject or a portion thereof (e.g., prostate, pituitary or mammary gland). For example, cell death may be imaged or detected using surgical or needle biopsy of a subject after administration of the annexin to the subject; or by the use of a catheter that may detect radiation in a vessel of a subject.

As used herein, the term “administering” to a subject includes dispensing, delivering or applying a composition of the invention to a subject by any suitable route for delivery of the composition to the desired location in the subject, including delivery by either the parenteral or oral route, intramuscular injection, subcutaneous/intradermal injection, intravenous injection, buccal administration, transdermal delivery and administration by the rectal, colonic, vaginal, intranasal or respiratory tract route.

The compositions of the invention may be administered to a subject in an amount effective, at dosages and for periods of time necessary, to achieve the desired result. An effective amount of the compositions of the invention may vary according to factors such as disease state, e.g., the tumor stage, age, and weight of the subject, and the ability of the composition to elicit a desired response in the subject. An effective amount is also one in which any toxic or detrimental effects (e.g., side effects) of the compositions are outweighed by the therapeutically or diagnostically beneficial effects. The compositions of the invention may be administered at a concentration of, for example, 1-1000 μg protein/kg, 1-900 μg protein/kg, 1-800 μg protein/kg, 1-700 μg protein/kg, 1-600 μg protein/kg, 1-500 μg protein/kg, 1-400 μg protein/kg, 1-300 μg protein/kg, 1-200 μg protein/kg, 1-100 μg protein/kg, 10-100 μg protein/kg, 10-80 μg protein/kg, 10-60 μg protein/kg, 10-40 μg protein/kg, or 10-20 μg protein/kg.

As used herein, the term “disease associated with cell death” is well known in the art and includes any disease or disorder associated with or caused by cell death. Examples of such diseases include, without limitation, tumorogenic diseases (e.g., cancers), organ and bone marrow transplant rejection or injury, infectious and non-infectious inflammatory diseases, autoimmune disease, arthritis, cerebral and myocardial infarction and ischemia, cardiomyopathies, atherosclerative disease, neural and neuromuscular degenerative diseases, sickle cell disease, β-thalassemia, AIDS, myelodysplastic syndromes, toxin-induced liver disease, and the like.

The magnetic reasonance image may be obtained using any of the art known techniques, for example, using a Picker Corp. Whole Body Superconducting System operating at 0.3 T using a 30 cm transmitter coil tuned to 0.26 T (10.08 MHz) or other MRI devices with field strengths ranging from 0.05 Tesla to 4.0 Tesla. Typically, the subject is placed in a powerful, highly uniform, static magnetic field. Magnetized protons (hydrogen nuclei) within the subject align like small magnets in this field. Radiofrequency pulses are then utilized to create an oscillating magnetic field perpendicular to the main field, from which the nuclei absorb energy and move out of alignment with the static field, in a state of excitation. As the nuclei return from excitation to the equilibrium state, a signal induced in the receiver coil of the instrument by the nuclear magnetization can then be transformed by a series of algorithms into images. Images based on different tissue characteristics can be obtained by varying the number and sequence of pulsed radiofrequency fields in order to take advantage of magnetic relaxation properties of the tissues.

If it is desired to follow the localization and/or the signal over time, for example, to record the effects of a treatment on the distribution and/or localization of cell death, the imaging can be repeated at selected time intervals to construct a series of images. The intervals can be as short as minutes, or as long as days, weeks, months or years. Images generated by methods of the present invention may be analyzed by a variety of methods. They range from a simple visual examination, mental evaluation and/or printing of a hardcopy, to sophisticated digital image analysis.

The magnetic reasonance image may be obtained 5, 10, 15, 20, 25, 30, 35, 40, 45, 50, 55, 60, 65, 70, 75, 80, 85, 90, 95, 100, 105, 110, 115, or 120 minutes after the administration of the magnetic reasonance imaging composition to the subject. In another embodiment, the magnetic reasonance image is obtained about 10-30, 15-25, 20-25, or 20-30 hours after the administration of the magnetic reasonance imaging composition to the subject. In a preferred embodiment, the magnetic reasonance image is obtained at a plurality of time points, thereby monitoring changes in the number of cells undergoing cell death or monitoring changes in the location of cells undergoing cell death.

The present invention also provides an optical imaging composition which includes an annexin or a fragment thereof, e.g., annexin V or a fragment thereof, coupled to an optically active molecule.

As used herein, an “optically active molecule” includes any molecule that has the ability to be optically detected, for example, by the use of medically available visualization devices such as endoscopes, bronchoscopes and minimally invasive surgical devices using optical detection of anatomic structures. According to the methods of the invention, the optically active molecule emits electromagnetic radiation in the visible (e.g., 400-700 nm) and/or near infrared (e.g., 700-1500 nm) portions of the electromagnetic spectrum. In one embodiment, the optically active molecule must be detectable using relatively simple mechanisms that do not require the transformation of non-visible electromagnetic radiation (e.g., gamma rays or X-rays) into a visible image, but that can be directly visualized. Examples of optically detectable molecules include fluorescein and methylene blue. Optically active molecules may also include those agents useful in photodynamic therapy (PDT). PDT works by exposing an annexin molecule linked to a photosensitizing molecule to specific wavelengths of light in the presence of oxygen. When this reaction occurs, the normally innocuous photosensitizing drug becomes cytotoxic via an activated species of oxygen, known as “singlet oxygen.” Examples of optically active agents which could be used in PDT when linked to annexin include PHOTOFRIN®, Lutrin, ANTRIN®, FOSCAN®, aminolevulinic acid, aluminum (III) phthalocyanine tetrasulfonate, Hypericin, verteporfin, and methylene blue dye.

In another aspect, the present invention provides a composition comprising an annexin or a fragment thereof, e.g., annexin V or a fragment thereof, coupled with a therapeutic radioisotope. As used herein, a “therapeutic radioisotope” is a radioisotope that is recognized as being useful and suitable for injection into a patient for therapeutic applications. A therapeutic radioisotope, as used herein, is preferably an alpha (α) or beta (β) emitting radioisotope. In one embodiment, the therapeutic radioisotope is not a gamma (γ) emitting radioisotope. Examples of therapeutic radioisotopes include ¹⁰³ Pd, ¹⁸⁶Re, ¹⁸⁸ Re, ⁹⁰Y, ¹⁵³Sm, ¹⁵⁹Gd, or ¹⁶⁶Ho, ¹³¹I, ¹²³I, ¹²⁶I, ¹³³I, ¹¹¹In, ¹⁷⁷Lu and ¹¹³In.

The therapeutic radioisotope and the annexin may be coupled at a ratio of 1:1, 1.1:1, 1.2:1, 1.3:1, 1.4:1, 1.5:1, 1.6:1, 1.7:1, 1.8:1, 1.9:1, or 2:1 (therapeutic radioisotope:annexin). Ranges of values using a combination of any of the above recited values as upper and/or lower limits are intended to be included in the present invention.

In a further aspect, the present invention provides a method of tumor radiotherapy by administering to a mammalian subject having a tumor an effective tumor reducing amount of a composition comprising an annexin coupled with a therapeutic radioisotope. The foregoing method may be used in conjunction with total body irradiation or targeted external irradiation and/or a treatment employing at least one chemotherapeutic agent (e.g., dimethyl busulfan, cyclophosphamide, bischloroethyl nitrosourea, cytosine arabinoside, or 6-thioguanine). The appropriate timing for the administration of the annexin-therapeutic isotope composition may be determined using any of the imaging techniques described herein or the imaging techniques described in U.S. Pat. No. 6,197,278 B1, the contents of which are incorporated herein by reference.

Various aspects of the invention are described in further detail in the following subsections:

Synthesis of Annexin Containing Compounds of the Invention

The invention can be practiced using purified native, recombinant, or synthetically-prepared annexin. Annexin V, for example, may be conveniently purified from human placenta (as described in Funakoshi, et al. (1987) Biochemistry 26:5572, the contents of which are incorporated herein by reference). Recombinant annexin offers several advantages, however, including ease of preparation and economic efficiency. A number of different annexins have been cloned from humans and other organisms. Their sequences are available in sequence databases, including GenBank.

The invention is preferably practiced using annexin V, for several reasons. First, annexin V is one of the most abundant annexins, (ii) it is simple to produce from natural or recombinant sources, and (iii) it has a high affinity for phospholipid membranes. Human annexin V has a molecular weight of 36 kd and a high affinity (kd=7 nmol/L) for phosphatidylserine (PS). The sequence of human annexin V can be obtained from GenBank under accession numbers U05760-U05770.

An exemplary expression system suitable for making annexin for use with the present invention employs the pET12a expression vector (Novagen, Madison, Wis.) in E. coli. (described in Wood, et al. (1996) Blood 88:1873-1880, incorporated herein by reference).

Other bacterial expression vectors may be utilized as well. They include, e.g., the plasmid pGEX (Smith, et al. (1988) Gene 67:31) and its derivatives (e.g., the pGEX series from Pharmacia Biotech, Piscataway, N.J.). These vectors express the polypeptide sequences of a cloned insert fused in-frame with glutathione-S-transferase. Recombinant pGEX plasmids can be transformed into appropriate strains of E. coli and fusion protein production can be induced by the addition of IPTG (isopropyl-thio galactopyranoside). Solubilized recombinant fusion protein can then be purified from cell lysates of the induced cultures using glutathione agarose affinity chromatography according to standard methods (described in, for example, Ausubel, et al. Current Protocols in Molecular Biology (John Wiley and Sons, Inc., Media, Pa.). Other commercially-available expression systems include yeast expression systems, such as the Pichia expression kit from Invitrogen (San Diego, Calif.); baculovirus expression systems (Reilly, et al. in Baculovirus Expression Vectors: A Laboratory Manual (1992); Clontech, Palo Alto Calif.); and mammalian cell expression systems (Clontech, Palo Alto Calif.; Gibco-BRL, Gaithersburg Md.).

A number of features can be engineered into the expression vectors, such as leader sequences which promote the secretion of the expressed sequences into culture medium. The recombinantly produced polypeptides are typically isolated from lysed cells or culture media.

Isolated recombinant polypeptides produced as described above may be purified by standard protein purification procedures, including differential precipitation, molecular sieve chromatography, ion exchange chromatography, isoelectric focusing, gel electrophoresis and affinity chromatography. Protein preparations can also be concentrated by, for example, filtration (Amicon, Danvers, Mass.).

Annexin produced as described above may then be coupled to a contrast agent, an optically active molecule, or a therapeutic radioisotope. The particular contrast agent, optically active molecule, or therapeutic radioisotope selected will depend on the particular application the skilled artisan intents to use.

Annexin Radiolabeling

Annexins may be radiolabeled by a variety of methods known in the art (e.g., as described in U.S. Pat. No. 5,985,240; U.S. Pat. No. 4,361,544 and U.S. Pat. No. 4,427,646, the entire contents of each of which are incorporated herein by reference). Annexins may be directly radioiodinated, through electrophilic substitution at reactive aromatic amino acids. Iodination may also be accomplished via pre-labeled reagents, in which the reagent is iodinated and purified, and then linked to the annexin. Iodination may also be achieved through the use of chelates, e.g., DTPA and EDTA chelates, as described in, for example, U.S. Pat. No. 4,986,979; U.S. Pat. No. 4,479,930 and U.S. Pat. No. 4,668,503.

In selecting a suitable therapeutic radioisotope, the skilled artisan will typically consider factors including, but not limited to, (i) minimum of particle emission, (ii) primary photon energy of between about 50 and 500 kEv, (iii) physical half-life greater that the time required to prepare material for administration [Iodine 123 (half-life of ˜13.2 hours), Iodine 131 (half-life of ˜8 days), Gallium 67 (half-life of ˜78 hours), and Indium 111 (half-life of ˜2.8 days)], (iv) effective half life longer than the examination time, suitable chemical form and reactivity, low toxicity, and stability or near stability of annexin labeled with that radioisotope.

Coupling of Annexin to Contrast Agents

Coupling of annexin to contrast agents may be performed using any of the art known techniques, e.g., chemical chelation techniques.

Coupling of annexin to a metal oxide may be performed as described in Chelating Agents and Metal Chelates, Dwyer & Mellor, Academic Press (1964), Chapter 7 and U.S. Pat. No. 5,443,816, the contents of each of which are incorporated herein by reference. Ionic forms of the elements iron, cobalt, nickel, copper, zinc, arsenic, selenium, technetium, ruthenium, palladium, silver, cadmium, indium, antimony, rhenium, osmium, iridium, platinum, gold, mercury, thallium, lead, bismuth, polonium and astatine may be used.

For example, annexin may incubated with a first reducing agent, the period of incubation being sufficient to reduce available disulfide bonds to thiolate groups while preventing excessive fragmentation of the annexin; the first reducing agent may then be substantially removed from the thiolate-containing annexin; a source of Sn (II) agent may then be added to the thiolate-containing annexin in a sufficient amount to form Sn (II)-containing and sulfur-containing complexes; and the Sn (II)-containing and sulfur-containing complexes may be labeled by adding the metal oxide, whereby the metal oxide displaces the Sn (II) agent and the metal oxide and thiolate-containing annexin form a complex. The order of the foregoing steps may be altered. For example, it is possible, and in some cases advantageous, to add the Sn (II) to form Sn (II)-containing and sulfur-containing complexes prior to removing excess reducing agent from the thiolate-containing annexin. In this way, oxidation of thiolate groups or reformation of disulfide bonds and other cross-linkages can be minimized.

A compound of the invention may be created by associating annexin with biodegradable superparamagnetic metal oxides such as iron oxide. Annexin associated with superparamagnetic or paramagnetic contrast agents provides the advantage of directing the magnetic resonance contrast agent to those cells which are apoptotic or necrotic. A compound prepared from annexin and biodegradable superparamagnetic iron oxide, for example, binds to hepatocytes which are rendered apoptotic by treatment with fas. A magnetic resonance experiment or imaging procedure carried out after administration to a subject of the compounds of the invention can, thus, provide a method for obtaining an enhanced magnetic resonance image, as well as valuable information regarding the distribution of damaged cells in the organism.

The use of magnetic particles for the attachment of biomolecules has been described by Molday (U.S. Pat. No. 4,452,773, the entire contents of which are incorporated herein by reference). Briefly, a dextran coated magnetic particle is formed and then treated with periodate to produce aldehyde groups. The aldehydes react with amino groups on a biological molecule, to form a Schiff base. The Schiff base may be stabilized by treatment with a reducing agent like sodium borohydride. After treatment with a reducing agent a methylene amino linker connects the biomolecule to the nanoparticle.

Other methods of attaching biomolecules to nanoparticles, which use the reactivity of the aldehyde group, may also be used, including the methods of Rembaum and Owen (see Table I).

The development of amine functionalized crosslinked iron oxide nanoparticle is another method of synthesizing magnetic particle-biomolecule conjugates that may be used to attach annexins to a metal oxide particle. Amino-CLIO is prepared by first synthesizing a dextran coated magnetic nanoparticle, followed by crosslinking the dextran with epichlorohydrin. Amine groups are incorporated by reacting the dextran with ammonia.

Table I (below) summarizes the types of magnetic particles that may be used for the attachement of Annexins, e.g., Annexin V.

TABLE I
Magnetic Particles That Can Be Used for the Attachment of Annexins

| | | Attachment Chem/ | | |
|---|---|---|---|---|
| Particle Size | | Biomolecule Attached | Polymer | Reference |
| <100 | nm | Periodate/antibody | Dextran | Abts (1989) J. Immunol Methods |
| | | | | 125, 19. |
| 10-70 | nm/ | Periodate/antibody | Dextran | U.S. Pat. No. 4,452,773 |
| | dextran | | | (Molday); Molday, (1982) |
| | | | | J. Immunol. Methods 52, 353. |
| 10-200 | nm/ | SPDP/antibody | BSA | U.S. Pat. No. 4,795,698 (Owen). |
| | albumin | | | |
| 10-50 | nm | Periodate/Synaptotagmin 1 | Carboxy Dextran | Zhoe (2001) Nat. Med. 7, 1241. |
| 40 | nm | SPDP.Oligonucleotides | Crosslinked | Josephson (1999) Bioconjug. |
| | | and Peptides | Dextran | Chem. 10, 186. |
| 10-200 | nm | Aldehydes/Enzymes, | Polyglutaraldehyde | U.S. Pat. No. 4,438,239 |
| | | Biomolecules | Polymer | (Rembaum) |
| | | | | U.S. Pat. No. 4,369,226. |
| 10-100 | nm | Periodate/Antibody | Dextran | U.S. Pat. No. 5,492,814 |
| | | | | (Weissleder) |

The conjugation of annexins to magnetic molecules yields materials that can be used in a variety of fields such as magnetic affinity chromatography, magnetic cell sorting, magnetic immunoassay and as MR imaging contrast agents. The requirements of the particle vary greatly with the intended application. For imaging applications, the magnetic particle must have a series of properties including:
(1) Size and size homogeneity. Magnetic particles are preferably smaller than the size of red blood cells (about 10 microns) to avoid clogging capillary beds. To achieve efficient targeting to a target cell or organ after injection, they must preferably be in the nanoparticle size range (1-500 nm). Larger particles are rapidly withdrawn from the vascular compartment by the phagocytic cells of the reticuloendothelial system, limiting their ability to react with a limited number of sites on the desired target. Magnetic particles preferably have a narrow size distribution, i.e., cannot have a small percentage of large particles which can occlude capillaries.
(2) Biodegradability. To be useful as a clinical diagnostic tool, magnetic particles must preferably be broken down and excreted or broken down and utilized by the body. Materials like polystyrene, while useful in the synthesis of magnetic particles for cell sorting, cannot be used in parenteral, clinical applications. The most common type of particle used for imaging applications are polymer coated iron oxides, with dextran or modified dextran being most often employed (Anzai (1994) Radiology 192, 709-15; Reimer (1995) Radiology 195, 489; Stark (1988) Radiology 168, 297).
(3) Safety. The magnetic particles must be non-toxic. Typically, the safety factor (the dose used for imaging divided by the dose killing 50% of a group of animals) is greater than 100 and preferably greater than 1000. Toxicity includes not only the generation of reversible or irreversible tissue damage, but also the induction of transient but annoying physiological reactions in selected subjects (such as humans) taking the preparation. These include fever, uticaria, mild pain, vomiting, and the like. To be useful as a clinical diagnostic agent, such as an MR imaging agent, the magnetic particle must preferably produce no discernable physiological response, except for the desired diagnostic information, in individuals taking preparation.
(4) Stability. To be used as a parenteral agent, the particle must preferably maintain its size distribution during a storage period, which, for pratical commercial reasons, is typically longer than 6 months and preferably as long as two years. Instability, evident as the growth in the number of large particles in the preparation, can result in particle induced toxicity, and the abrupt end to the commercial use of the product.

A wide variety of conjugating strategies have been employed to couple proteins to each other and can be adapted to couple Annexins, e.g., Annexin V to magnetic particles, as would be obvious to one skilled in the art. Many of these reagents consist of an N-hydroxysuccinimide ester, which reacts with an amine, and a second moiety that reacts with a sulfhydryl group. A wide selection of bifunctional conjugating reagents, such as SPDP, SMCC, SATA and SlAt are available from Piece Chemical Company. Detailed procedures for their use are available from the Piece Chemical web site (see http://www.piercenet.com).

Coupling of Annexin to Optically Active Molecules

Coupling of annexin to optically active molecules may be performed using any of the art known techniques, e.g., those described in U.S. Pat. No. 5,312,922; U.S. Pat. No. 5,928,627; U.S. Pat. No. 6,096,289; Weir, ed., Handbook of Experimental Immunology, Vol. 1, Chapter 28, pp. 28.1-28.21, Oxford, Blackwell Scientific, 1986, the entire contents of each of which are incorporated herein by reference.

Administration of the Annexin Containing Compounds of the Invention

The annexin containing compounds of the present invention may be administered to a subject using standard protocols, such as protocols for the administration of radiolabeled compounds.

The compositions of the invention may be administered to a subject in an amount effective, at dosages and for periods of time necessary, to achieve the desired result. An effective amount of the compositions of the invention may vary according to factors such as the tumor stage, age, and weight of the subject, and the ability of the composition to elicit a desired response in the subject. An effective amount is also one in which any toxic or detrimental effects (e.g., side effects) of the compositions are outweighed by the therapeutically or diagnostically beneficial effects. The compositions of the invention may be administered at a concentration of 10-1000 μg protein/kg, 10-900 μg protein/kg, 10-800 μg protein/kg, 10-700 μg protein/kg, 10-600 μg protein/kg, 10-500 μg protein/kg, 10-400 μg protein/kg, 10-300 μg protein/kg, 10-200 μg protein/kg, or 10-100 μg protein/kg.

Annexin V begins to have pharmacological effects (anti-coagulant effects) at doses greater than about 300 μg/kg. Accordingly, the diagnostic methods of the present invention (which seek to avoid pharmacological effects of the labeled annexin) are preferably practiced at doses lower than 300 μg/kg, typically less than about 50 μg/kg. Such tracer doses (e.g., 10 μg/kg to 50 μg/kg) have no reported pharmacologic or toxic side effects in animal or human subjects.

The compounds of the invention are typically suspended in a suitable delivery vehicle, such as sterile saline. The vehicle may also contain stabilizing agents, carriers, excipients, stabilizers, emulsifiers, and the like, as is recognized in the art.

The compounds of the invention may be administered to a subject by any suitable route for administration. A preferred method of administration is intravenous (i.v.) injection. It is particularly suitable for imaging of well-vascularized internal organs, such as the heart, liver, spleen, and the like. Methods for i.v. injection of, e.g., radiopharmaceuticals are known. For example, it is recognized that a radiolabeled pharmaceutical is typically administered as a bolus injection using either the Oldendorf/Tourniquet method or the intravenous push method (see, e.g., Mettler and Guierbteau, (1985) Essentials Of Nuclear Medicine Imaging, Second Edition, W.B. Saunders Company, Philadelphia, Pa.).

For imaging the brain, the compositions of the invention can be administered intrathecally. Intrathecal administration delivers a compound directly to the sub-arachnoid space containing cerebral spinal fluid (CSF). Delivery to spinal cord regions can also be accomplished by epidural injection to a region of the spinal cord exterior to the arachnoid membrane.

For bronchoscopy applications, the annexin compounds of the present invention may be administered by inhalation. For example, the annexin compounds may be delivered in the form of an aerosol spray from a pressured container or dispenser which contains a suitable propellant, e.g., a gas such as carbon dioxide, or a nebulizer.

Other modes of administration include intraperitoneal (e.g., for patients on kidney dialysis), and intrapleural administration. For specific applications, the invention contemplates additional modes of delivery, including intramuscular injection, subcutaneous, intralymphatic, insufflation, and oral, intravaginal and/or rectal administration.

Localization of the Annexin Containing Compounds of the Invention

After the compounds of the invention are administered, they are allowed to localize to the target tissue or organ. Localization in this context refers to a condition when either an equilibrium or a pseudo-steady state relationship between bound, “localized”, and unbound, “free” compound within a subject has been achieved. The amount of time required for such localization is typically on the order of minutes to tens of minutes and may be estimated by the serum half-life of the compound. The localization time also depends on the accessibility of the target tissue to the compound. This, in turn, depends on the mode of administration, as is recognized in the art.

Imaging is preferably initiated after most of the compound has localized to its target(s). For intravenously administered Tc99m-labeled annexin V, this occurs after several half-lives. A duration of about 10 half-lives (about 30-240 min in the case of annexin/Tc99m conjugates) is considered to be ample time to achieve essentially complete localization. One of skill in the art will appreciate, however, that it may be desirable to perform the imaging at times less than or greater than the ˜10 half-life timepoint described above. For example, in imaging cell death due to blood vessel injury, the accessibility of the target tissue is very high, such that a strong signal can be obtained from the target site in only a few minutes, especially if a low dose of labeled annexin is administered gradually to minimize signal from circulating label.

In all of the above cases, a reasonable estimate of the time to achieve localization may be made by one skilled in the art. Furthermore, the state of localization as a function of time may be followed by imaging the gamma ray signal from the labeled annexin according to the methods of the invention.

Applications

Major uses for the annexin containing compounds of the invention include the detection of inappropriate apoptosis in disease states where it should not occur, e.g., immune disorders such as Lupus, transplant rejection, or in cells subject to severe ischemia; and the detection of insufficient apoptosis when it should occur, e.g., tumors or cells infected with a virus.

The annexin containing compounds of the invention may be employed in a variety of clinical settings in which apoptotic and/or necrotic cell death need to be monitored, such as, without limitation, organ and bone marrow transplant rejection or injury, infectious and non-infectious inflammatory diseases, autoimmune disease, cerebral and myocardial infarction and ischemia, cardiomyopathies, atherosclerative disease, neural and neuromuscular degenerative diseases, sickle cell disease, β-thalassemia, cancer therapy, AIDS, myelodysplastic syndromes, and toxin-induced liver disease, and the like. The annexin containing compounds of the invention may also be useful as a clinical research tool to study the normal immune system, embryological development, and immune tolerance and allergy.

The compounds of the invention can be used, for example, to image and quantify apoptotic cell death in normal and malignant tissues undergoing treatment. Monitoring apoptosis with serial imaging studies using these compounds can be used for the rapid testing and development of new drugs and therapies in a variety of diseases. In addition, the methods may be used to monitor the progress of treatment, monitor the progress of disease, or both. Further, they may be used to aid in early detection of certain diseases.

An advantage of the above method is that, by imaging at selected intervals, the method can be used to track changes in the intensity of the emission from the subject over time, reflecting changes in the number of cells undergoing cell death. Such an approach may also be used to track changes in the localization of the compounds of the invention in the subject over time, reflecting changes in the distribution of cells undergoing cell death.

The compositions and methods of the present invention may also be used in the diagnosis and/or treatment of subjects suffering from an eye disease, such as, for example, retinal disease or glaucoma.

The photodynamic therapy (PDT) methods disclosed herein are particularly useful for treating a range of diseases characterized by rapidly growing tissue, including the formation of abnormal blood vessels, such as cancer and age-related macular degeneration (AMD). The type of light source used in PDT varies according to the condition treated. For example, for opthalmology applications, diode laser light may be shone through the slit lamp of a microscope into a subject's eye. For cancer/internal diseases, fiber optics may be used to deliver light to the internal cavities like the lung, the gastro-intestinal tract and esophagus and light-emitting diodes (LED) may be used for skin cancer.

In summary, the compositions and methods of the present invention provide a number of clinical and diagnostic benefits. For example, using the methods of the invention, the response of individual patients to established therapeutic anti-cancer regimens may be efficiently and timely evaluated; the anti-neoplastic activity of new anti-cancer drugs may be evaluated; the optimal dose and dosing schedules for new anti-cancer drugs may be identified; and the optimal dose and dosing schedules for existing anti-cancer drugs and drug combinations may be identified. In addition, using the methods of the invention, cancer patients in clinical trials may be categorized efficiently into responders and non-responders to therapeutic regimens.

The methods of the invention provide, among other things, a non-invasive technique for evaluating the early response of individual patient tumors to chemotherapy. This facilitates the selection of effective treatment by allowing rapid identification of ineffective treatments whose side effects might not be balanced by expected benefits.

Prognostic Applications

The present invention also provides methods for predicting the response of a subject having a disease associated with cell death, such as a tumorogenic disease, to a therapeutic regimen (e.g., chemotherapy or radiotherapy). In particular, by way of the present invention, it was discovered that the uptake of annexin by an area of cell death, e.g., a tumor, prior to treatment may be used as an in vivo biomarker for predicting the response of the subject to a therapeutic regimen for treating the disease associated with the cell death. Accordingly, prognostic applications of the invention can be used to predict therapeutic responses for a variety of diseases associated with cell death including, but not limited to, tumorogenic diseases (cancers), autoimmune diseases and infections diseases.

In one embodiment, the method includes administering to the subject an annexin or fragment thereof which is detectably labeled, and detecting the localization of the annexin or fragment thereof within the subject. The localization of the labeled annexin, or fragment thereof, within the area of cell death, e.g., within the tumor or in the region of the tumor, is indicative of a positive response by the subject to the therapeutic regimen. The area of cell death may be inspected (e.g., visually or quantitatively) for the uptake of label using methods well known in the art and described herein. For example, a CT (computed tomography) scan of the area of cell death may be obtained prior to administration of the labeled annexin, followed by obtaining a nuclear scan of the area of cell death subsequent to administration of the labeled annexin. The two images then may be overlaid (a process also known in the art as “registration” of the image) and inspected for the presence of label. The registration process may be performed by, for example, a computer using software such as the Visualization Data Explorer™ (IBM Corporation). Positron Emission Tomography (PET) may also be used, as is well known in the art. An increase of label in the area of cell death, e.g., the area of the tumor, as compared to that in normal tissue (the background) would indicate that the subject under examination has an increased probability of responding to therapy. Alternatively, when the label in the area of cell death, e.g., the area of the tumor, is not increased as compared to normal tissue (the background), then the subject under examination is less likely to respond to treatment.

The labeled annexin or fragment thereof may be administered to the subject as described herein and the localization of the labeled annexin may be determined either immediately or after several hours or days depending on the particular application. For example, localization can be determined between about 0.1-72 hours, 0.1-48 hours, 0.1-36 hours, 0.1-24 hours, 1-20 hours, 1-15 hours, 2-10 hours, 2-8 hours, 4-8 hours or, preferably, between about 4-6 hours after the administration of the labeled annexin to the subject. Ranges intermediate to the above recited values, e.g., 0.2-2 hours, 0.2-1 hours, 12-24 hours, 24-36 hours or 6-24 hours, also can be used. For example, ranges using a combination of any of the above recited values as upper and/or lower limits can be used.

The invention also provides methods for using the uptake of annexin by an area of cell death, e.g., a tumor, as an in vivo biomarker of early response, e.g., tumor response, to a therapeutic regimen (e.g., chemotherapy or radiotherapy). The methods include administering to the subject a therapeutic regimen; administering to the subject (e.g., after or at the same time as the administration of the therapeutic regimen) an annexin or fragment thereof which is detectably labeled, and detecting a change in the uptake of the annexin or fragment thereof by the area of cell death, e.g., the tumor, over time. A change in the uptake of the annexin or fragment thereof by the area of cell death, e.g., the tumor, over time is indicative of a positive response by the subject to the therapeutic regimen. The change may be an increase or, in some embodiments, a decrease in the uptake of the annexin or fragment thereof by the area of cell death, e.g., the tumor. The change may be detected, for example, by comparing the uptake of the annexin or fragment thereof by the area of cell death, e.g., the tumor, before and after the administration of the therapeutic regimen or by comparing the uptake of the annexin or fragment thereof by the area of cell death, e.g., the tumor, at different time points after the administration of the therapeutic regimen. Images of the area of cell death, e.g., the tumor, may be obtained, as described above, prior to and between about 0.1-72 hours, 0.1-48 hours, 0.1-36 hours, 0.1-24 hours, 1-20 hours, 1-15 hours, 2-10 hours, 2-8 hours, 4-8 hours or, preferably, between about 4-6 hours following the administration of the therapeutic regimen, e.g., following the initiation of chemotherapy. The labeled annexin may be detected between about 0.1-72 hours, 0.1-48 hours, 0.1-36 hours, 0.1-24 hours, 1-20 hours, 1-15 hours, 2-10 hours, 2-8 hours, 4-8 hours or, preferably, between about 4-6 hours after the administration of the labeled annexin to the subject. Alternatively, if the labeled annexin is to be administered at multiple times, the label may be detected between about 0.1-72 hours, 0.1-48 hours, 0.1-36 hours, 0.1-24 hours, 1-20 hours, 1-15 hours, 2-10 hours, 2-8 hours, 4-8 hours or, preferably, between about 4-6 hours following each labeled annexin administration. Ranges intermediate to the above recited values, e.g., 0.2-2 hours, 0.2-1 hours, 12-24 hours, 24-36 hours or 6-24 hours, are also intended to be part of this invention. For example, ranges using a combination of any of the above recited values as upper and/or lower limits are intended to be included.

For the prognostic applications of the invention, any suitable label may be used to label the annexin or fragment thereof. For example, a contrast agent or an optically active molecule may be used (as described herein) or a radioisotope may be used (as described in, for example, U.S. Pat. No. 6,197,278 B1, the contents of which are incorporated herein by reference). The particular radioisotope for coupling with the annexin or fragment thereof will depend on the particular method being used. The invention may be practiced with any one of a variety of radioisotopes presently available. In selecting a suitable radioisotope, the practitioner will typically consider the particular application of the invention, along with factors common to nuclear imaging in general. Such factors include: (i) minimum of particle emission, (ii) primary photon energy of between about 50 and 511 kEv, (iii) physical half-life greater than the time required to prepare material for administration, (iv) effective half life longer than the examination time, suitable chemical form and reactivity, low toxicity, and stability or near stability of the annexin or fragment thereof labeled with that radioisotope. In a preferred embodiment, ^{99m}Technetium (Tc99m) is used. Tc99m has a half-life of about 6 hours and can be used to label annexin or a fragment thereof to high specific activities. It fulfills most of the above criteria and is used in over 80% of nuclear medicine imaging procedures. Generally positron emitting and single photon-emitting radioisotopes may be utilized in various embodiments of the present invention (Kung et al., 1993). Exemplary isotopes that may be used include, but are not limited to, ¹²³Iodine (half-life of ˜13.2 hours), ¹³¹Iodine (half-life of ˜8 days), ⁶⁷Gallium (half-life of ˜78 hours), ¹⁸Fluorine (half-life of ˜110 minutes), ¹¹¹Indium (half-life of ˜2.8 days), ⁶⁸Gallium, ⁸⁹Zirconium and ¹⁷⁷Lutetium. Methods for labeling the annexin or fragment thereof are well known in the art and described herein as well as in U.S. Pat. No. 6,197,278 B1 and U.S. Provisional Patent Application Ser. Nos. 60/504,118 and 60/506,638, the contents of each of which are incorporated herein by reference.

The invention is further illustrated by the following examples, which should not be construed as further limiting. The contents of all references, pending patent applications and published patents, cited throughout this application, as well as the Figures are hereby expressly incorporated by reference.

### EXAMPLES

### Example 1

### Attachment Of Annexin V To Dextran Coated Magnetic Iron Oxides Through The Use Of Periodate

Periodate treatment of the dextran coated magnetic particle produces an aldehyde, which forms a Schiff base with the amines of the Annexin V. The complex is stabilized by treatment with sodium borohydride.

A dextran coated superparamagnetic iron oxide nanoparticle was synthesized according to the methods of Molday (1982) J. Immunol. Methods 52, 353. Iron oxide (10 mg Fe in about 1 mL of water) and purified Annexin V were dialyzed against sodium acetate (0.01M, pH 6). Annexin V was purified by the method of Wood (1996) Blood 88, 1873. The amount of Annexin V can be varied from 1 to about 50 mg, preferably 5-10 mg of protein. At lower amounts the ratio of protein to iron on the resulting magnetic nanoparticle will be lower, but the offered protein will couple more efficiently. At higher amounts of protein, the ratio of protein to iron on the resulting nanoparticle will be higher, but the percent of protein coupled will be lower.

Freshly made sodium periodate (50 mg/mL, 0.2 mL) was added to the iron oxide. The mixture was then incubated for 30 minutes at room temperature in the dark, and dialyzed against 0.15 M NaGI. The oxidized magnetic iron oxide was then mixed with the Annexin V and the pH adjusted by the addition of 100 μl of 0.2 M sodium bicarbonate, pH 9.5. The mixture was incubated for 3 hours with stirring. Freshly made sodium cyanoborohydride was then added (25 mg/mL, 0.2 mL) and the mixture was incubated for 6 hours at room temperature. The Annexin V-magnetic nanoparticle can be separated from the unreacted Annexin by a variety sized based separation methods. These include gel filtration, ultrafiltration or magnetic separation.

### Example 2

### Attachment of Annexin V with a Sulfhydryl Group to Amino CLIO

The amino-CLIO nanoparticle was made as described in Josephson (1999) Bioconjug. Chem. 10, 186. Annexin V with a sulfhydryl group added through mutagenesis (Tait (2000) Bioconjug Chem 11, 918) was employed. To 1.2 mL of amino-CLIO in (30 mg Fe) was added 1.2 mL of 0.1 M phosphate buffer, pH 7.4, and 2 mL of N-succinimidyl 3-(2-pyridyldithio)propionate (SPDP, 25 mM) (Molecular Bio-sciences, Boulder, Colo.) in DMSO. The mixture was allowed to stand for 60 minutes at room temperature. Low molecular impurities were removed by PD-10 columns (Sigma Chemical, St- Louis, Mo.) equilibrated with 0.01M Tris and 0.02 M citrate, pH 7.4 buffer.

Between 2 and 50 mg of Annexin V was subsequently added to 10 mg Fe of the SPDP activated nanoparticle at room temperature and the mixture was allowed to stand overnight. The Annexin V-magnetic nanoparticle can be separated from the unreacted annexin by a variety sized based separation methods.

### Example 3

### Reaction of Annexin V to Add a Sulfhydryl Group, Followed by Reaction with Amino CLIO

A sulfhydryl group was added to the annexin (obtained as in Example 1) by use of the reagent SATA following the manufacturers instructions, Pierce Chemical Company. Amino-CLIO was reacted with SPDP as in Example 2 and then reacted with the SAT A reacted annexin.

### Example 4

### Attachment of Annexin V to a BSA Coated Magnetic Particle

BSA coated magnetic particles were made as described in U.S. Pat. No. 4,795,698. Some of the amine groups of the BSA coating of the magnetic particle are converted to sulfydryl groups by use of the reagent SPDP (see Example 2). SPDP or SATA can then be used to add one or more sulfydryl groups on Annexin V. After treatment of the Annexin V with DTT, to expose a sulfhydryl group, the protein is reacted with the magnetic particle.

### Example 5

### Chemotherapy Induced Change in ^{99m}TC-HYNIC-RH-Annexin V Uptake as an Early Predictor of Response to Platinum Therapy in Advanced Non-Small-Cell Lung Cancer

The effectiveness of most currently available anti-tumor agents is believed to depend upon their ability to induce apoptosis in susceptible tumor cells. Imaging with ^{99m}Tc-Annexin V was evaluated for the in vivo assessment of tissue apoptosis and necrosis. To assess ^{99m}Tc-Annexin V tissue localization as a biomarker of early response to anti-tumor treatment, early post-chemotherapy changes in ^{99m}Tc-Annexin V tissue distribution have been evaluated as an early predictor of response to platinum-based chemotherapy in patients with stage IIIB/IV Non-Small-Cell Lung Cancer (NSCLC).

Planar and SPECT images of 24 patients with stage IIIB or IV NSCLC were obtained prior to, and between 12 and 24 hours following, initiation of chemotherapy. Scintigraphic images were obtained 4-6 hours following each 15-25 mCi ^{99m}Tc-Hynic-rh-Annexin V administration. Nuclear images were evaluated for visual change (increase or decrease) in Annexin localization in the region of the lung tumor by an experienced reader with access to baseline CT images, but blinded to clinical response data. Another experienced radiologist blinded to the Annexin results and to clinical data independently determined objective response from baseline and 6-12 week post-chemotherapy chest CT scans using standard RECIST criteria.

Seven of 24 subjects (29%) had a partial response to treatment; none had a complete response. Seven of 7 responders (100%) showed a change in uptake of ^{99m}Tc-Annexin (6 increased, 1 decreased). No subject with unchanged ^{99m}Tc-Annexin uptake showed a morphometric response. Of the 17 patients not responding to chemotherapy, 15 showed no change in localization and 2 showed a decrease (see Table I). In this study, change in ^{99m}Tc-Annexin uptake within 24 hours of initiation of chemotherapy predicted chemotherapy response with 100% sensitivity and 88% specificity. The positive predictive value (PPV) was 78% and the negative predictive value (NPV) 100%.

In view of the foregoing results, it is evident that treatment-induced change in ^{99m}Tc-Annexin V localization within 24 hours of initiation of the first course of platinum-based chemotherapy in patients with advanced NSCLC was associated with a marked increase in the likelihood of objective response.

TABLE I
Relationship between change in Annexin uptake and chemotherapy
response (RECIST)

| | Responders | Non-Responders | Total |
|---|---|---|---|
| Change | 7 | 2 | 9 |
| No Change | 0 | 15 | 15 |
| Total | 7 | 17 | 24 |

### Example 2

### Pre-Treatment Uptake of ^{99m}Tc Annexin V is Associated with an Increased Platinum-Based Chemotherapy Response Rate in Advanced Non-Small-Cell Lung Cancer

Apoptotic Index (AI)—the percentage of apoptotic cells—has been evaluated as a prognostic marker in human tumors. Imaging with ^{99m}Tc-Annexin V is under evaluation as a non-invasive technique to assess tumor apoptosis in situ. In this study, the utility of baseline, pre-treatment uptake of ^{99m}Tc-Annexin V as a biomarker for response to platinum-based chemotherapy was assessed in patients with stage IIIB/IV Non-Small-Cell Lung Cancer (NSCLC).

In a multi-center phase II study of patients with stage IIIB and IV NSCLC undergoing platinum-based chemotherapy, planar and SPECT images of the chest were obtained between four and six hours following administration of 15-25 mCi ^{99m}Tc-Hynic-rh-Annexin V. Baseline images obtained within five days prior to administration of the first dose of chemotherapy were evaluated for the presence of focal uptake of Annexin in the region of the lung tumor by an experienced reader with access to the baseline chest CT, but blinded to clinical response data. Objective chemotherapy response using standard RECIST criteria was determined from baseline and 6-12 week post-chemotherapy chest CT scans by a second, independent reader blinded to the Annexin scans and to clinical data.

Seven of 24 patients (29%) demonstrated a partial response to treatment. All 7 responders (100%) showed focal Annexin uptake in the region of their tumor. Of the 17 patients not responding to chemotherapy, 9 (53%) showed no uptake (see Table II). In this study, baseline Annexin uptake as a biomarker predictive of chemotherapy response had a sensitivity of 100%, a specificity of 53%, a positive predictive value of 47%, and a negative predictive value of 100%.

In view of the foregoing results, it is evident that pre-treatment uptake of ^{99m}Tc-Annexin V is a novel biomarker for response prediction in patients with stage IIIB/IV NSCLC undergoing platinum-based chemotherapy.

TABLE II
Relationship between baseline (pre-chemotherapy) Annexin uptake and
chemotherapy response (RECIST)

| | Present | Absent | Total |
|---|---|---|---|
| Responders | 7 | 0 | 7 |
| Non-Responders | 8 | 9 | 17 |
| Total | 15 | 9 | 24 |

Equivalents

Those skilled in the art will recognize, or be able to ascertain using no more than routine experimentation, many equivalents to the specific embodiments of the invention described herein. Such equivalents are intended to be encompassed by the following claims.

1. A method for predicting the response to a therapeutic regimen in a subject having a disease associated with cell death, the method comprising
administering to said subject an annexin or fragment thereof which is detectably labeled, and
detecting the localization of said annexin or fragment thereof within said subject, wherein the presence of said annexin or fragment thereof in the region of cell death is indicative of a positive response by said subject to the therapeutic regimen.

2. The method of claim 1, wherein said subject is human.

3. The method of claim 1, wherein said therapeutic regimen comprises chemotherapy.

4. The method of claim 3, wherein said chemotherapy involves administration of a chemotherapeutic agent selected from the group consisting of dimethyl busulfan, cyclophosphamide, bischloroethyl nitrosourea, cytosine arabinoside, and 6-thioguanine.

5. The method of claim 1, wherein said therapeutic regimen comprises platinum-based chemotherapy.

6. The method of claim 1, wherein said therapeutic regimen comprises administration of an apoptosis inducing agent selected from the group consisting of TNF, TRAIL and Fas.

7. The method of claim 1, wherein said therapeutic regimen comprises administration of an apoptosis inducing agent selected from the group consisting of a TNF-binding antibody, a TRAIL-binding antibody and a Fas-binding antibody.

8. The method of claim 1, wherein said therapeutic regimen comprises total body irradiation or targeted external irradiation.

9. The method of claim 1, wherein said therapeutic regimen comprises targeted internal irradiation.

10. The method of claim 1, wherein said therapeutic regimen comprises total body irradiation or targeted external irradiation and the administration of a chemotherapeutic agent.

11. The method of claim 1, wherein said annexin comprises annexin V or a fragment thereof.

12. The method of claim 11, wherein said annexin V fragment comprises a phospholipid binding domain of annexin V.

13. The method of claim 11, wherein said annexin V fragment comprises domain 1 of annexin V.

14. The method of claim 1, wherein said annexin comprises an annexin derivative.

15. The method of claim 14, wherein said annexin derivative comprises an annexin V derivative.

16. The method of claim 1, wherein said annexin comprises a small molecule wherein the small molecule mimics domain 1 of annexin V.

17. The method of claim 1, wherein said annexin comprises recombinantly produced annexin.

18. The method of claim 1, wherein said annexin or fragment thereof is administered via a method selected from the group consisting of intraperitoneally, intrathecally, intrapleurally, intralymphatically and intramuscularly.

19. The method of claim 1, wherein said annexin or fragment thereof is administered intravenously.

20. The method of claim 1, wherein said annexin or fragment thereof is administered at a concentration of 1-500 μg protein/kg.

21. The method of claim 1, wherein said annexin or fragment thereof is administered at a concentration of 1-200 μg protein/kg.

22. The method of claim 1, wherein said annexin or fragment thereof is detectably labeled using a contrast agent.

23. The method of claim 22, wherein the detecting step comprises obtaining a magnetic resonance image.

24. The method of claim 1, wherein said annexin or fragment thereof is detectably labeled using a radioisotope.

25. The method of claim 24, wherein said radioisotope is selected from the group consisting of ¹²³Iodine, ¹³¹Iodine, ⁶⁷Gallium, ¹¹¹Indium, ¹⁸Fluorine, ^{99m}Technetium (Tc99m), ⁶⁸Gallium, and ⁸⁹Zirconium.

26. The method of claim 25, wherein said radioisotope comprises Tc99m.

27. The method of claim 26, wherein said Tc99m is linked to said annexin or fragment thereof via hydrazino nicotinamide (HYNIC).

28. The method of claim 24, wherein the detecting step comprises measuring radiation emission from said radioisotope in said subject with a radiation detector device, thereby constructing an image of radiation emission.

29. The method of claim 28, wherein said radiation detector device is a gamma ray detector device and the radiation emission is gamma ray emission.

30. The method of claim 29, wherein said gamma ray detector device is a gamma scintillation camera.

31. The method of claim 28, wherein said radiation detector device is a 3-dimensional imaging camera.

32. The method of claim 1, wherein said annexin or fragment thereof is detectably labeled using an optically active molecule.

33. The method of claim 32, wherein said optically active molecule comprises a fluorescent dye.

34. The method of claim 32, wherein the detecting step comprises illuminating said subject with a light source and visually monitoring the presence of the detectable label.

35. The method of claim 1, wherein the detecting step is performed between about 4 to about 6 hours after said administration of said annexin or fragment thereof.

36. The method of claim 1, wherein the detecting step is performed between about 4 to about 12 hours after said administration of said annexin or fragment thereof.

37. The method of claim 1, wherein said disease is a tumor.

38. The method of claim 37, wherein said detecting comprises overlaying a CT scan and a nuclear scan of said tumor.

39. The method of claim 37, wherein the tumor is present in an organ of a subject or a portion thereof.

40. The method of claim 37, wherein the tumor is present in the lung of a subject or a portion thereof.

41. The method of claim 40, wherein said subject is suffering from advanced non-small-cell lung cancer.

42. The method of claim 37, wherein the tumor is present in an area of a subject selected from the group consisting of the head of a subject or a portion thereof, the colon of a subject or a portion thereof, the heart of a subject or a portion thereof, the liver of a subject or a portion thereof, the eye of a subject or a portion thereof, the breast of a subject or a portion thereof, the prostate of a subject or a portion thereof and the stomach of a subject or a portion thereof.

43. The method of claim 37, wherein the tumor is present in the gastrointestinal tract of a subject.

44. The method of claim 37, wherein the tumor is present in the breast of a subject.

45. The method of claim 37, wherein the tumor is lymphoma.

46. The method of claim 37, wherein the tumor is present in the prostate of a subject.

47. The method of claim 1, wherein said disease is an autoimmune disease.

48. The method of claim 1, wherein said disease is arthritis.

49. A method for predicting the response to a therapeutic regimen in a subject having a disease associated with cell death, the method comprising
administering to said subject a therapeutic regimen;
administering to said subject an annexin or fragment thereof which is detectably labeled, and
detecting a change in the uptake of said annexin or fragment thereof by the area of cell death over time, wherein a change in the uptake of said annexin or fragment thereof by said area of cell death over time is indicative of a positive response by said subject to said therapeutic regimen.

50. The method of claim 49, wherein said change is an increase in the uptake of said annexin or fragment thereof by said area of cell death over time.

51. The method of claim 49, wherein said change is a decrease in the uptake of said annexin or fragment thereof by said area of cell death over time.

52. The method of claim 49, wherein said change is detected by comparing the uptake of said annexin or fragment thereof by said area of cell death before and after the administration of said therapeutic regimen.

53. The method of claim 49, wherein said change is detected by comparing the uptake of said annexin or fragment thereof by said area of cell death at different time points after the administration of said therapeutic regimen.

54. The method of claim 49, wherein said therapeutic regimen and said annexin or fragment thereof are co-administered to said subject.

55. A method for predicting the response to tumortherapy in a subject having a tumor, the method comprising
administering to said subject an annexin or fragment thereof which is detectably labeled, and
detecting the localization of said annexin or fragment thereof within said subject, wherein the presence of said annexin or fragment thereof in the region of said tumor or within said tumor is indicative of a positive response by said subject to a therapeutic regimen.
